(19) United States
(12) Patent Application Publication
(10) Pub. No.: US 2025/0346248 A1
(43) Pub. Date: Nov. 13, 2025

(54) SYSTEM AND METHOD FOR REDUCING MOTION SICKNESS

(71) Applicant: HYUNDAI MOBIS CO., LTD., Seoul (KR)

(72) Inventors: Ki Hee PARK, Yongin-si (KR); Ji Soo SHIN, Yongin-si (KR)

(73) Assignee: HYUNDAI MOBIS CO., LTD., Seoul (KR)

(21) Appl. No.: 18/977,401

(22) Filed: Dec. 11, 2024

(30) Foreign Application Priority Data
May 8, 2024 (KR) 10-2024-0060502

(51) Int. Cl. B60W 50/16; B60K 35/28; B60W 40/08; B60W 50/00; B60W 50/14; G06V 20/59; G06V 40/10

(52) U.S. Cl. CPC B60W 50/16; B60K 35/28; B60W 40/08; B60W 50/0098; G06V 20/59; G06V 40/10; B60K 2360/177; B60K 2360/1868; B60W 2040/0872; B60W 2050/146; B60W 2420/403; B60W 2520/105; B60W 2520/14; B60W 2520/16; B60W 2520/18; B60W 2540/18; B60W 2540/221

(57) ABSTRACT

A system for reducing motion sickness of a passenger in a moving device and a method therefor are provided. The system includes a state information acquisition unit to collect state information of the passenger, a behavior information acquisition unit to collect real-time driving information of the moving device from at least one sensor of the moving device, a processor to determine a motion sickness state of the passenger based on the collected state information and the collected real-time driving information, and generate motion sickness reduction information for reducing motion sickness of the passenger based on the determined motion sickness state; and a driving unit to provide visual or tactile perception of the passenger based on the generated motion sickness reduction information.

### CROSS-REFERENCE TO RELATED APPLICATION(S)

This application claims the benefit under 35 USC § 119 of Korean Patent Application No. 10-2024-0060502 filed on May 8, 2024, in the Korean Intellectual Property Office, the entire disclosures of which is hereby incorporated by reference for all purposes.

### BACKGROUND

### 1. Field

The present disclosure relates to a system and method for reducing motion sickness capable of reducing motion sickness of a passenger riding in a moving device in a special environment using visual recognition or tactile stimulation.

### 2. Description of the Related Art

Motion sickness may occur when exposed to one or more specific motions for a long period of time. In this instance, factors such as temperature, smell, emotions, and digestion may serve to promote motion sickness.

In particular, many people experience car sickness, and many solutions have been proposed to suppress car sickness. A representative example thereof is a suspension taken before riding in a vehicle, but this suspension contains ingredients such as scopolamine, dimenhydrinate, diphenhydramine, promethazine, and meclizine, and thus have many side effects.

Accordingly, anti-motion sickness patches are mainly used these days. However, since anti-motion sickness patches do not have the same effect on everyone and in all situations, consumers are greatly dissatisfied with effectiveness thereof.

Motion sickness, which is accompanied by dizziness and nausea when riding in a vehicle, is caused by the brain temporarily becoming confused when there is a mismatch in input between sensory organs (visual, somatosensory, semicircular canal, etc.) that maintain balance or detect movement and posture.

A human remembers responses of sensory organs such as eyes and ears to muscle movement in the brain, and prepares for and responds to similar movement thereafter by prediction of the sensory organs using remembered information. However, in a state of riding in a vehicle, there is no muscle movement due to moving, or movement is different from existing memory, so that a mismatch in sensation occurs and motion sickness occurs.

As a conventional technology, motion sickness detection technology only uses extremely limited vibration information of a vehicle, and thus has a problem of not being able to accurately detect motion sickness of a passenger.

Therefore, to reduce motion sickness of a vehicle passenger, a means is needed to reduce motion sickness of the vehicle passenger by reducing a difference between movement actually felt by the passenger and movement perceived by a brain of the passenger with respect to real-time driving information of a vehicle.

### SUMMARY

The present disclosure is to address at least the above-mentioned problems and/or disadvantages and to provide at least the advantages described below. Accordingly, an aspect of the present disclosure is to provide a system and method for reducing motion sickness, and more specifically, a system and method for reducing motion sickness capable of reducing motion sickness of a passenger riding in a moving device in a special environment using visual recognition or tactile stimulation.

Another aspect of the present disclosure is to provide a system and method for reducing motion sickness capable of reducing motion sickness of a vehicle passenger by reducing a difference between movement actually felt by the passenger and movement perceived by a brain of the passenger.

Another aspect of the present disclosure is to provide a system and method for reducing motion sickness capable of reducing motion sickness of a passenger riding in a special vehicle movable on land and sea.

The problems to be solved by the present disclosure are not limited to the technical problems mentioned above, and other technical problems not mentioned herein may be clearly understood by a person having ordinary skill in the technical field to which the present disclosure pertains from the description below.

In a general aspect of the disclosure, a system for reducing motion sickness of a passenger in a moving device, includes: a state information acquisition unit configured to collect state information of the passenger; a behavior information acquisition unit configured to collect real-time driving information of the moving device from at least one sensor of the moving device; a processor configured to: determine a motion sickness state of the passenger based on the collected state information and the collected real-time driving information; and generate motion sickness reduction information for reducing motion sickness of the passenger based on the determined motion sickness state; and a driving unit located in the moving device and configured to provide visual or tactile perception of the passenger based on the generated motion sickness reduction information.

The state information acquisition unit may be further configured to collect state information of each of a plurality of passengers riding in the moving device, and the processor may be further configured to determine a motion sickness state of each of the plurality of passengers based on the collected state information, and individually generate the motion sickness reduction information.

The driving unit may include: a display configured to output the real-time driving information of the moving device so that the real-time driving information is visually recognizable by the passenger; and an air conditioner located below the display and configured to blow air toward the passenger.

The motion sickness reduction information generated by the processor may include an augmented reality image that reflects the real-time driving information of the moving device, wherein the display outputs the augmented reality image.

Each of a plurality of displays may be arranged in one direction, wherein each of the plurality of displays is individually driven.

Each of a plurality of air conditioners may be arranged in one direction, wherein each of the plurality of air conditioners may be individually driven.

The plurality of air conditioners may change at least one of a direction, temperature, or intensity of the air being ejected.

The state information acquisition unit may include a wearable biosensor allowed to be worn by the passenger, wherein the biosensor may acquire and monitor a biosignal of at least one of EEG, heart rate, electrocardiogram, or pulse of the passenger.

The state information acquisition unit may include a camera located in the moving device, wherein the state information acquisition unit may be further configured to photograph and monitor position and temperature changes of the passenger.

The moving device may include a vehicle that is movable on land and water.

The at least one sensor of the moving device may include at least one of an acceleration sensor, a brake sensor, a tilt sensor, a yaw/pitch/roll sensor, a steering angle sensor, a GPS sensor, or any combination thereof.

In another general aspect of the disclosure, a method of reducing motion sickness of a passenger in a moving device, includes: collecting state information of the passenger; collecting real-time driving information of the moving device from at least one sensor located in the moving device; determining a motion sickness state of the passenger based on the collected state information and the collected real-time driving information; generating motion sickness reduction information for reducing motion sickness of the passenger based on the determined motion sickness state; and providing visual or tactile perception of the passenger based on the generated motion sickness reduction information.

The collecting of the state information may include collecting state information of each of a plurality of passengers riding in the moving device, wherein the generating of the motion sickness reduction information may include: determining a motion sickness state of each of the plurality of passengers based on the collected state information; and individually generating the motion sickness reduction information.

In yet another general aspect of the disclosure, a system for reducing motion sickness of a passenger in a vehicle, may include: a first sensor configured to collect state information of the passenger; a second sensor configured to collect real-time driving information of the moving device; a display in the moving device; and a processor configured to: determine a motion sickness state of the passenger based on the collected state information and the collected real-time driving information; generate motion sickness reduction information for reducing motion sickness of the passenger based on the determined motion sickness state; and control the display to provide visual or tactile perception of the passenger based on the generated motion sickness reduction information.

The vehicle may include at least one of an off-road vehicle, a tracked vehicle, a hovercraft, or an aircraft.

The first sensor may include at least one of a biosensor, a wearable sensor, a camera, or any combination thereof.

The second sensor may include at least one of an acceleration sensor, a brake sensor, a tilt sensor, a yaw/pitch/roll sensor, a steering angle sensor, a GPS sensor, or any combination thereof.

### BRIEF DESCRIPTION OF THE DRAWINGS

The accompanying drawings, which are included to provide a further understanding of the disclosure and are incorporated in and constitute a part of this application, illustrate embodiment(s) of the disclosure and together with the description serve to explain the principle of the disclosure. In the drawings:

FIG. 1 is a block diagram of a motion sickness reduction system according to an embodiment of the present disclosure;

FIG. 2 is a diagram illustrating a wearable biosensor in the motion sickness reduction system according to an embodiment of the present disclosure;

FIG. 3 is a diagram illustrating an inside of a moving device in the motion sickness reduction system according to an embodiment of the present disclosure;

FIG. 4 is a diagram illustrating a display and an air conditioner of a driving unit in the motion sickness reduction system according to an embodiment of the present disclosure;

FIG. 5 is a diagram for describing the air conditioner in the motion sickness reduction system according to an embodiment of the present disclosure;

FIG. 6 is a diagram illustrating an augmented reality image output through the display in the motion sickness reduction system according to an embodiment of the present disclosure;

FIG. 7 is a diagram for describing driving of the driving unit in the motion sickness reduction system according to an embodiment of the present disclosure; and

FIG. 8 is a diagram for describing a motion sickness reduction method according to an embodiment of the present disclosure.

### DETAILED DESCRIPTION

Description will now be given in detail according to exemplary embodiments disclosed herein, with reference to the accompanying drawings. The same or equivalent components may be provided with the same reference numbers, and description thereof will not be repeated. As used herein, the suffixes “module” and “part” are added or used interchangeably to facilitate preparation of this specification and are not intended to suggest distinct meanings or functions. In describing embodiments disclosed in this specification, relevant well-known technologies may not be described in detail in order not to obscure the subject matter of the embodiments disclosed in this specification. In addition, it should be noted that the accompanying drawings are only for easy understanding of the embodiments disclosed in the present specification, and should not be construed as limiting the technical spirit disclosed in the present specification. As such, the present disclosure should be construed to extend to any alterations, equivalents and substitutes in addition to those which are particularly set out in the accompanying drawings.

Although the terms first, second, etc. may be used herein to describe various elements, these elements should not be limited by these terms. These terms are generally only used to distinguish one element from another.

It will be understood that when an element is referred to as being “connected with” another element, the element can be directly connected with the other element or intervening elements may also be present. In contrast, it will be understood that when an element is referred to as being “directly connected with” another element, there are no intervening elements present.

A singular representation may include a plural representation unless it represents a definitely different meaning from the context.

The terms such as “include” or “have” used herein are intended to indicate that features, numbers, steps, operations, elements, components, or combinations thereof used in the following description exist and it should be thus understood that the possibility of existence or addition of one or more different features, numbers, steps, operations, elements, components, or combinations thereof is not excluded.

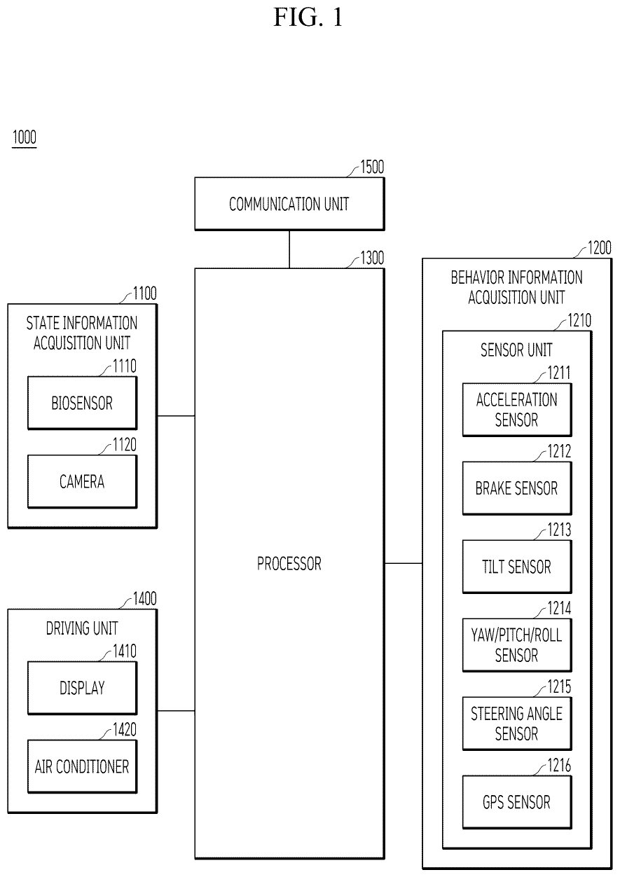

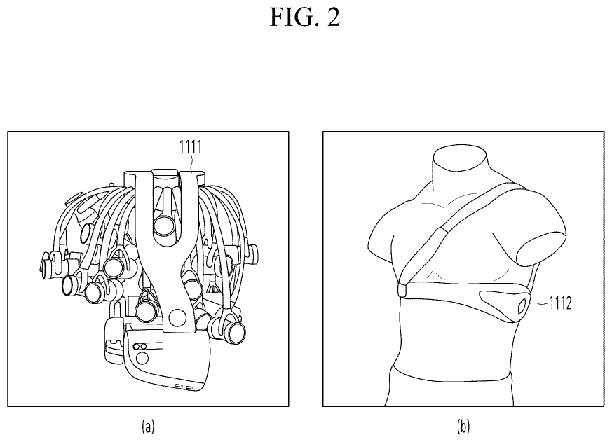

FIG. 1 is a block diagram of a motion sickness reduction system 1000 according to an embodiment of the present disclosure. In addition, FIG. 2 is a diagram illustrating a wearable biosensor 1110 in the motion sickness reduction system 1000 according to an embodiment of the present disclosure.

The motion sickness reduction system 1000 according to an embodiment of the present disclosure may include a state information acquisition unit 1100, a behavior information acquisition unit 1200, a processor 1300, a driving unit 1400, and a communication unit 1500.

First, the state information acquisition unit 1100 may include a biosensor 1110 and a camera 1120. Here, the biosensor 1110 may be a wearable biosensor that may be worn by a passenger. In addition, the biosensor 1110 may include an electroencephalogram (EEG) sensor, an electrocardiogram sensor, a skin conductance sensor, and a respiration detection sensor.

For example, as illustrated in FIG. 2A, the biosensor 1110 may include a wearable sensor 1111 that may be worn on a head of the passenger to measure an EEG signal. In addition, as illustrated in FIG. 2B, the biosensor 1110 may include a wearable sensor 1112 that may be worn by the passenger to measure a heart rate signal.

In addition, the biosensor 1110 may include a smartwatch for measuring a photoplethysmogram (PPG) signal. The smartwatch may be worn on a wrist of the passenger and may measure a biosignal such as heart rate using a blood flow of the passenger.

Further, the biosensor 1110 described above may be used to acquire a biosignal of at least one of EEG, heart rate, electrocardiogram, or pulse of the passenger. In addition, an image of the passenger may be monitored through the camera 1120 to collect state information such as location of the passenger and temperature change.

That is, the motion sickness reduction system 1000 according to an embodiment of the present disclosure may collect state information of the passenger through the state information acquisition unit 1100.

The communication unit 1500 may wirelessly transmit state information of the passenger collected through the above-described state information acquisition unit 1110 to the processor 1300 using communication such as Bluetooth, infrared communication, RFID, or UWB, or may transmit the state information of the passenger to the processor 1300 by wire. In addition, the communication unit 1500 may be connected wirelessly or by wire to various devices carried by the passenger.

The behavior information acquisition unit 1200 may collect real-time driving information of a moving device from sensors located in the moving device. Here, a sensor unit 1210 may include an acceleration sensor 1211, a brake sensor 1212, a tilt sensor 1213, a yaw/pitch/roll sensor 1214, a steering angle sensor 1215, and a GPS sensor 1216.

That is, the motion sickness reduction system 1000 according to an embodiment of the present disclosure may collect real-time driving information of the moving device, such as straight driving, turning, changes in speed, acceleration, and changes in height, through the sensor unit 1210 described above.

As described above, motion sickness, which is accompanied by dizziness and vomiting when riding in the moving device, is caused by a temporary confusion in the brain when there is a mismatch in input between the sensory organs (visual, somatosensory, semicircular canal, etc.) that maintain balance or detect movement and posture. In humans, reactions of the sensory organs such as the eyes and ears to muscle movements are remembered in the brain, and when similar movement occurs later, the sensory organs predict in advance and prepare and react based on the remembered information.

However, in a state of riding in the moving device, there is no muscle movement due to moving, or movement is different from the existing memory, and thus sensory mismatch occurs, causing motion sickness of the passenger of the moving device. Here, there was a problem that motion sickness of the passenger could not be accurately detected since only vibration information of the moving device, which was extremely limited, was used to detect motion sickness in the past.

Accordingly, an object of the motion sickness reduction system 1000 according to an embodiment of the present disclosure is to reduce motion sickness of the passenger of the moving device by reducing a difference between movement actually felt by the passenger and movement recognized by the brain of the passenger with respect to real-time driving information of the moving device through the state information acquisition unit 1100 that collects state information of the passenger and the behavior information acquisition unit 1200 that collects real-time driving information of the moving device.

In particular, in the motion sickness reduction system 1000 according to an embodiment of the present disclosure, the moving device may include a special vehicle movable on land and sea. For example, the special vehicle may be a military armored vehicle. In this case, due to characteristics of the special vehicle moving in a special environment, a passenger riding in the special vehicle may experience more severe motion sickness than a passenger riding in a general vehicle.

Accordingly, the motion sickness reduction system 1000 according to an embodiment of the present disclosure is intended to provide a means for reducing motion sickness of the passenger in a special environment, and more specific details thereof will be described later.

In the motion sickness reduction system 1000 according to an embodiment of the present disclosure, the processor 1300 may determine a motion sickness state of the passenger based on state information collected through the state information acquisition unit 1100 and real-time driving information collected through the behavior information acquisition unit 1200, and may generate motion sickness reduction information for reducing motion sickness of the passenger based on the determined motion sickness state.

Further, the driving unit 1400 located in the moving device may be driven to allow visual or tactile perception of the passenger based on the motion sickness reduction information generated through the processor 1300.

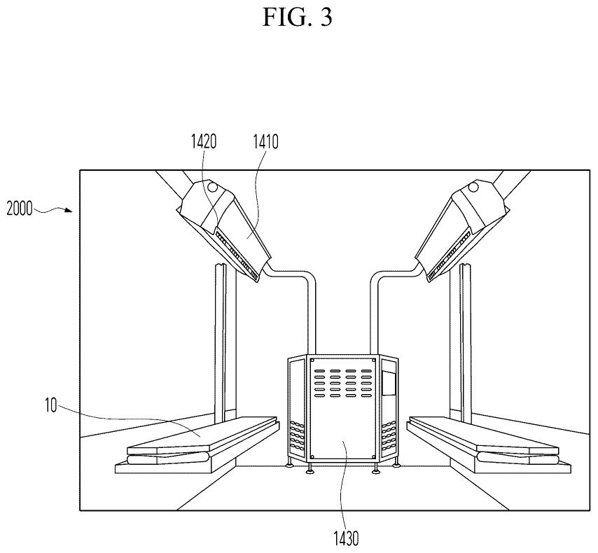

More specifically, referring to FIG. 3, the driving unit 1400 may include a display 1410 that outputs real-time driving information of the moving device 2000 so that the passenger may visually recognize the real-time driving information. In addition, the driving unit 1400 may include an air conditioner 1420 located below the display 1410 to blow air toward the passenger. A structure and operation of the driving unit 1400 will be described later.

Meanwhile, the block diagram of the motion sickness reduction system 1000 illustrated in FIG. 1 is merely a block diagram for an embodiment of the present disclosure, and each component of the block diagram may be integrated, added, or omitted according to the specifications of the motion sickness reduction system 1000 that is actually implemented. That is, two or more components may be combined into one component, or one component may be divided into two or more components and configured, as needed. In addition, a function performed by each block is for describing an embodiment of the present disclosure, and a specific operation or device thereof does not limit the scope of rights of the present disclosure.

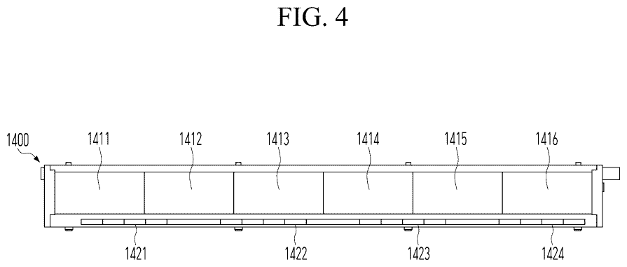

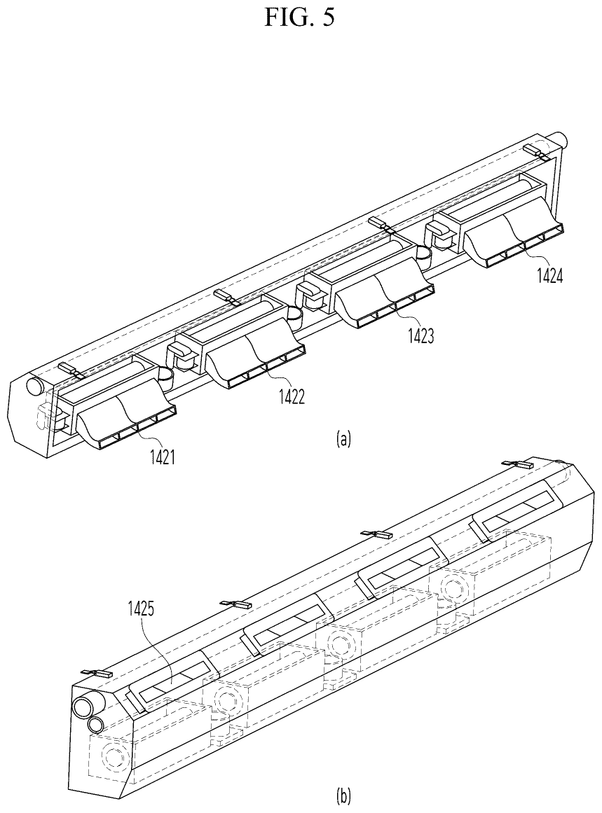

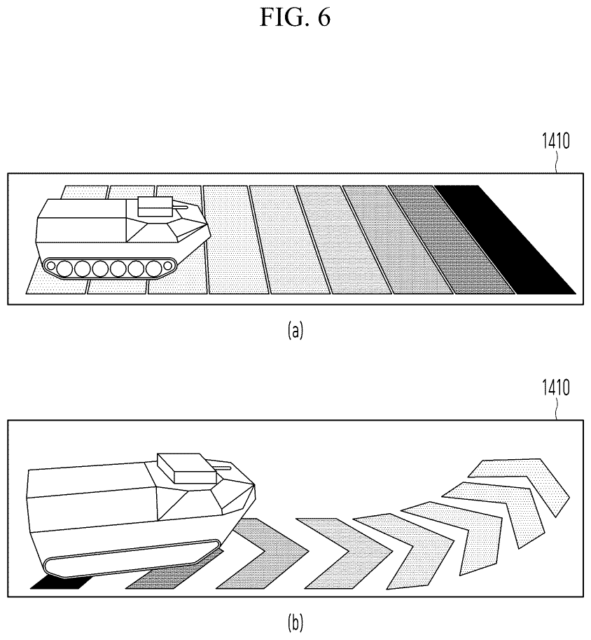

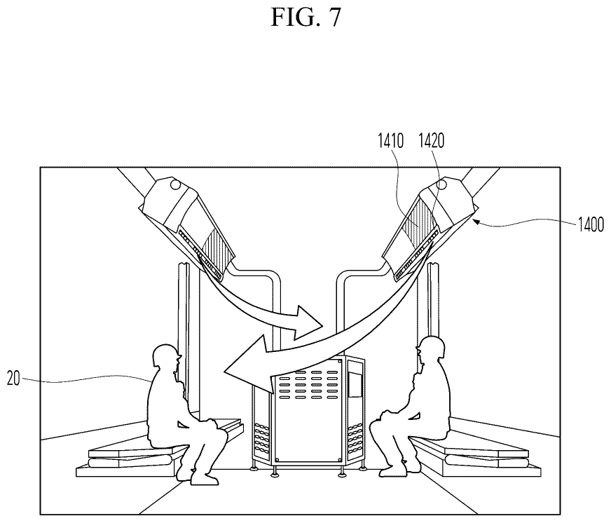

FIG. 3 is a diagram illustrating an inside of the moving device 2000 in the motion sickness reduction system 1000 according to an embodiment of the present disclosure. FIG. 4 is a diagram illustrating the display 1410 and the air conditioner 1420 of the driving unit 1400 in the motion sickness reduction system 1000 according to an embodiment of the present disclosure. FIG. 5 is a diagram for describing the air conditioner 1420 in the motion sickness reduction system 1000 according to an embodiment of the present disclosure. FIG. 6 is a diagram illustrating an augmented reality image output through the display 1410 in the motion sickness reduction system 1000 according to an embodiment of the present disclosure. Further, FIG. 7 is a diagram for describing driving of the driving unit 1400 in the motion sickness reduction system 1000 according to an embodiment of the present disclosure.

As described above, in the motion sickness reduction system 1000 according to an embodiment of the present disclosure, the moving device 2000 may include a special vehicle movable on land and sea. For example, the special vehicle may be a military armored vehicle. As illustrated in FIG. 3, the military armored vehicle may include a pair of seats 10 facing each other and arranged in one direction. Further, as illustrated in FIG. 7, a plurality of passengers 20 may ride on the pair of seats 10.

Here, the state information acquisition unit 1100 described above through FIG. 1 may collect state information of each of the plurality of passengers 20 riding in the moving device 2000. Then, the processor 1300 may determine a motion sickness state of each of the plurality of passengers 20 based on the collected state information and individually generate motion sickness reduction information.

That is, the motion sickness reduction system 1000 according to an embodiment of the present disclosure may reduce motion sickness of the plurality of individual passengers 20 riding in a special vehicle operated in a special environment through individual visual recognition or tactile stimulation.

More specifically, as illustrated in FIG. 4, in the motion sickness reduction system 1000 according to an embodiment of the present disclosure, the driving unit 1400 may include a plurality of displays 1411, 1412, 1413, 1414, 1415, and 1416 arranged in one direction. Here, each of the plurality of displays 1411, 1412, 1413, 1414, 1415, and 1416 may be individually driven.

Referring to FIG. 6, motion sickness reduction information generated by the processor 1300 may include an augmented reality image reflecting real-time driving information of the moving device 2000. In addition, the display 1410 may output the augmented reality image. FIG. 6A is an example of the case of moving straight, and FIG. 6B is an example of the case of moving while rotating.

In addition, each of the above-described plurality of displays 1411, 1412, 1413, 1414, 1415, and 1416 may individually output augmented reality images based on motion sickness reduction information individually generated for the plurality of passengers 20 via the processor 1300.

Further, in the motion sickness reduction system 1000 according to an embodiment of the present disclosure, the driving unit 1400 may include a plurality of air conditioners 1421, 1422, 1423, and 1424 arranged in one direction. Likewise, each of the plurality of air conditioners 1421, 1422, 1423, and 1424 may be individually driven.

In addition, each of the above-described plurality of air conditioners 1421, 1422, 1423, and 1424 may be individually driven based on motion sickness reduction information individually generated for each of the plurality of passengers 20 through the processor 1300 as illustrated in FIG. 7. Here, the plurality of air conditioners 1421, 1422, 1423, and 1424 may change at least one of a direction, temperature, or intensity of air being ejected.

Additionally, FIG. 5A is a drawing illustrating a structure in which the plurality of air conditioners 1421, 1422, 1423, and 1424 is installed in the driving unit 1400. In addition, FIG. 5B is a drawing illustrating an activated carbon filter 1425 arranged on a rear surface of the driving unit 1400. As described above, purification of air ejected from the air conditioner 1420 is required due to movement characteristics of the special vehicle, and the activated carbon filter 1425 may serve to allow purified air to be ejected from the air conditioner 1420.

In addition, the motion sickness reduction system 1000 according to an embodiment of the present disclosure may include a driving source 1430 that is installed in the moving device 2000 as illustrated in FIG. 3 and supplies power to and drives the driving unit 1400. Further, the display 1410 and the air conditioner 1420 of the driving unit 1400 described above may be driven through the driving source 1430.

Therefore, the motion sickness reduction system 1000 according to an embodiment of the present disclosure may provide individual care for a passenger 20 suffering from motion sickness based on state information of each of the plurality of passengers 20 riding in the moving device 2000 in a special environment. Further, to this end, as described above, a plurality of displays 1410 and a plurality of air conditioners 1420 may be individually operated to reduce motion sickness of the passenger 20 by enhancing visual recognition ability of the passenger 20 and performing tactile stimulation.

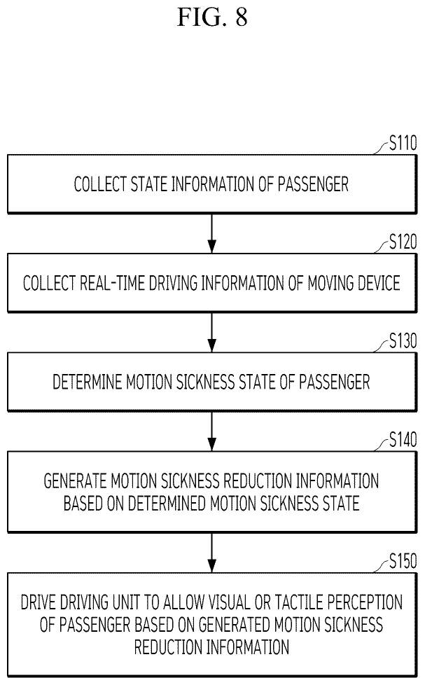

FIG. 8 is a diagram for describing a motion sickness reduction method according to an embodiment of the present disclosure.

Hereinafter, the motion sickness reduction method according to the embodiment of the present disclosure will be described by summarizing matters described above with reference to FIGS. 1 to 7.

First, state information of the passenger 20 may be collected through the state information acquisition unit 1100 (S110), and real-time driving information of the moving device 2000 may be collected from the sensors of the moving device 2000 (S120).

Thereafter, a motion sickness state of the passenger 20 may be determined based on the collected real-time driving information and the collected state information (S130), and motion sickness reduction information for reducing motion sickness of the passenger 20 may be generated based on the determined motion sickness state (S140). Then, based on the generated motion sickness reduction information, the driving unit 1400 may be driven to allow visual or tactile perception of the passenger 20 (S150).

In particular, in the motion sickness reduction method according to an embodiment of the present disclosure, the moving device 2000 may include a special vehicle movable on land and sea as described above. For example, the special vehicle may be a military armored vehicle. In this case, due to the characteristics of the special vehicle moving in a special environment, a passenger 20 riding in the special vehicle may experience more severe motion sickness than a passenger 20 riding in a general vehicle. In addition, as described above, the plurality of passengers 20 may ride in the special vehicle and move.

Accordingly, in the motion sickness reduction method according to an embodiment of the present disclosure, the step (S110) of collecting state information of the passenger 20 may include collecting state information of each of the plurality of passengers 20 riding in the moving device 2000. In addition, the step (S120) of generating motion sickness reduction information may include individually generating motion sickness reduction information by determining a motion sickness state of each of the plurality of passengers 20 based on the collected state information.

Further, in the motion sickness reduction method according to an embodiment of the present disclosure, as described above with reference to FIG. 5, the driving unit 1400 may include the plurality of individually driven displays 1411, 1412, 1413, 1414, 1415, and 1416 and the plurality of individually driven air conditioners 1421, 1422, 1423, and 1424.

Further, in this way, the motion sickness reduction method according to an embodiment of the present disclosure may provide individual care for a passenger 20 suffering from motion sickness based on state information of each of the plurality of passengers 20 riding in the moving device 2000 in a special environment.

To summarize the above description, the system and method for reducing motion sickness according to an embodiment of the present disclosure may reduce motion sickness of a passenger riding in a moving device in a special environment using visual recognition or tactile stimulation. In addition, the system and method may reduce motion sickness of a vehicle passenger by reducing a difference between movement actually felt by the passenger and movement perceived by a brain of the passenger. In addition, the system and method may reduce motion sickness of a passenger riding in a special vehicle movable on land and sea.

The system and method for reducing motion sickness according to an embodiment of the present disclosure may reduce motion sickness of a passenger riding in a moving device in a special environment using visual recognition or tactile stimulation.

In addition, the system and method may reduce motion sickness of a vehicle passenger by reducing a difference between movement actually felt by the passenger and movement perceived by a brain of the passenger.

In addition, the system and method may reduce motion sickness of a passenger riding in a special vehicle movable on land and sea.

The effects obtainable from the present disclosure are not limited to the effects mentioned above, and other effects not mentioned herein will be clearly understood by those skilled in the art to which the present disclosure pertains from the above description.

The above detailed description is to be construed in all aspects as illustrative and not restrictive. The scope of the present disclosure should be determined by reasonable interpretation of the appended claims and all changes coming within the equivalency range of the present disclosure are intended to be embraced in the scope of the present disclosure.

What is claimed is:

1. A system for reducing motion sickness of a passenger in a moving device, the system comprising:
a state information acquisition unit configured to collect state information of the passenger;
a behavior information acquisition unit configured to collect real-time driving information of the moving device from at least one sensor of the moving device;
a processor configured to:
determine a motion sickness state of the passenger based on the collected state information and the collected real-time driving information; and
generate motion sickness reduction information for reducing motion sickness of the passenger based on the determined motion sickness state; and
a driving unit located in the moving device and configured to provide visual or tactile perception of the passenger based on the generated motion sickness reduction information.

2. The system according to claim 1,
wherein the state information acquisition unit is further configured to collect state information of each of a plurality of passengers riding in the moving device, and
wherein the processor is further configured to determine a motion sickness state of each of the plurality of passengers based on the collected state information, and individually generate the motion sickness reduction information.

3. The system according to claim 1, wherein the driving unit comprises:
a display configured to output the real-time driving information of the moving device so that the real-time driving information is visually recognizable by the passenger; and
an air conditioner located below the display and configured to blow air toward the passenger.

4. The system according to claim 3,
wherein the motion sickness reduction information generated by the processor comprises an augmented reality image that reflects the real-time driving information of the moving device, and
wherein the display outputs the augmented reality image.

5. The system according to claim 3,
wherein each of a plurality of displays is arranged in one direction, and
wherein each of the plurality of displays is individually driven.

6. The system according to claim 5,
wherein each of a plurality of air conditioners is arranged in one direction, and
wherein each of the plurality of air conditioners is individually driven.

7. The system according to claim 6, wherein the plurality of air conditioners changes at least one of a direction, temperature, or intensity of the air being ejected.

8. The system according to claim 1,
wherein the state information acquisition unit comprises a wearable biosensor allowed to be worn by the passenger, and
wherein the biosensor acquires and monitors a biosignal of at least one of EEG, heart rate, electrocardiogram, or pulse of the passenger.

9. The system according to claim 1,
wherein the state information acquisition unit comprises a camera located in the moving device, and
wherein the state information acquisition unit is further configured to photograph and monitor position and temperature changes of the passenger.

10. The system according to claim 1, wherein the moving device comprises a vehicle that is movable on land and water.

11. The system according to claim 1, wherein the at least one sensor of the moving device comprises at least one of an acceleration sensor, a brake sensor, a tilt sensor, a yaw/pitch/roll sensor, a steering angle sensor, a GPS sensor, or any combination thereof.

12. A method of reducing motion sickness of a passenger in a moving device, the method comprising:
collecting state information of the passenger;
collecting real-time driving information of the moving device from at least one sensor located in the moving device;
determining a motion sickness state of the passenger based on the collected state information and the collected real-time driving information;
generating motion sickness reduction information for reducing motion sickness of the passenger based on the determined motion sickness state; and
providing visual or tactile perception of the passenger based on the generated motion sickness reduction information.

13. The method according to claim 12,
wherein the collecting of the state information comprises collecting state information of each of a plurality of passengers riding in the moving device, and
wherein the generating of the motion sickness reduction information comprises:
determining a motion sickness state of each of the plurality of passengers based on the collected state information; and
individually generating the motion sickness reduction information.

14. A system for reducing motion sickness of a passenger in a vehicle, the system comprising:
a first sensor configured to collect state information of the passenger;
a second sensor configured to collect real-time driving information of the moving device;
a display in the moving device; and
a processor configured to:
determine a motion sickness state of the passenger based on the collected state information and the collected real-time driving information;
generate motion sickness reduction information for reducing motion sickness of the passenger based on the determined motion sickness state; and
control the display to provide visual or tactile perception of the passenger based on the generated motion sickness reduction information.

15. The system of claim 14, wherein the vehicle comprises at least one of an off-road vehicle, a tracked vehicle, a hovercraft, or an aircraft.

16. The system of claim 14, wherein the first sensor comprises at least one of a biosensor, a wearable sensor, a camera, or any combination thereof.

17. The system of claim 14, wherein the second sensor comprises at least one of an acceleration sensor, a brake sensor, a tilt sensor, a yaw/pitch/roll sensor, a steering angle sensor, a GPS sensor, or any combination thereof.
